(19) United States
(12) Patent Application Publication
(10) Pub. No.: US 2024/0366127 A1
(43) Pub. Date: Nov. 7, 2024

(54) MICRO ANALYTE SENSOR AND CONTINUOUS ANALYTE MONITORING DEVICE

(71) Applicant: MEDTRUM TECHNOLOGIES INC., Shanghai (CN)

(72) Inventor: Cuijun Yang, Shanghai (CN)

(73) Assignee: MEDTRUM TECHNOLOGIES INC., Shanghai (CN)

(21) Appl. No.: 18/576,368

(22) PCT Filed: Jul. 8, 2021
(86) PCT No.: PCT/CN2021/105108

§ 371 Date: Jan. 4, 2024

(51) Int. Cl. A61B 5/1486; A61B 5/145

(52) U.S. Cl. CPC A61B 5/14865; A61B 5/14532

(57) ABSTRACT

A miniature analyte detection device, includes a base and at least two electrode groups. The base includes an internal part and an external part. The at least two electrode groups are located on the surface of the internal part, each electrode group includes at least one working electrode and at least one additional electrode. The external part is provided with a PAD corresponding to each electrode, and the PAD is electrically connected with the working electrode and the additional electrode respectively through a wire. The working electrode and the additional electrode are configured to trigger alternately into the working state according to the predetermined conditions when in use, so as to prolong the service life of the sensor, improve the service reliability and enhance the user experience.

### TECHNICAL FIELD

The invention mainly relates to the field of medical devices, in particular to a micro analyte sensor.

### BACKGROUND

The pancreas in a normal human body can automatically monitor the level of glucose in the human blood and automatically secrete the required insulin/glucagon. In diabetics, the pancreas does not function properly and cannot produce the insulin the body needs. Therefore, diabetes is a metabolic disease caused by abnormal pancreatic function, and diabetes is a lifelong disease. At present, there is no cure for diabetes with medical technology. The occurrence and development of diabetes and its complications can only be controlled by stabilizing blood glucose.

Diabetics need to have their blood glucose measured before they inject insulin into the body. At present, most of the testing methods can continuously measure blood glucose and send the data to a remote device in real time for the user to view. This method is called Continuous Glucose Monitoring (CGM). The method requires the device to be attached to the skin and the probe it carries is inserted into the tissue fluid beneath the skin.

But the current sensor on the enzyme activity of time limitation, so the service life of CGM device is often restricted to the service life of the sensor, in general, the service life of the sensor in 1˜14 days, after more than the service life of the enzyme activity decline, measured the parameters of the analyte data reliability will also decline, so in use after a certain period of time, the user needs to replace the new sensor, which causes inconvenience in the use and increases the cost of the user.

Therefore, the existing technology urgently needs a kind of micro analyte sensor with longer service life and higher reliability.

### BRIEF SUMMARY OF THE INVENTION

In view of the above the disadvantages of existing technology, the present invention implementation example first published in a micro analyte sensor, including at least two groups of electrodes, decorate in the sensor substrate, electrode group under the condition of presupposition triggers, alternately into the working state, prolong the service life of sensors, improve service reliability, enhance the user experience.

The invention discloses a micro analyte sensor, which comprises a base, the base comprises an internal part and an external part. At least two electrode groups, located on the surface of the internal part, each electrode group comprises at least one working electrode and at least one additional electrode. The external part is provided with a PAD corresponding to each electrode, and the PAD is electrically connected with the working electrode and the additional electrode respectively through a wire. The working electrode and the additional electrode are configured so that, when in use, they are triggered in accordance with predetermined conditions and enter the working state alternately.

According to one aspect of the invention, the additional electrode includes a counter electrode.

According to one aspect of the invention, the additional electrode also includes a reference electrode.

According to one aspect of the invention, the working electrode, the reference electrode and the counter electrode at least include an electron conduction layer, anti-interference layer, enzyme layer, adjustment layer and biological compatible layer.

According to one aspect of the invention, the electron conduction layer of the working electrode and the counter electrode is one of graphite, glassy carbon or precious metals.

According to one aspect of the invention, the electron conduction layer of the working electrode and the counter electrode is platinum.

According to one aspect of the invention, the electron conduction layer of the reference electrode is either Ag/AgCl or calomel.

According to one aspect of the invention, the enzyme layer is the glucose oxidase layer.

According to one aspect of the invention, the internal part is of a planar structure, and the working electrode, the reference electrode and the counter electrode are insulated from each other and laid flat on the surface of the internal part.

According to one aspect of the invention, the internal part has a step structure, and the working electrode, the reference electrode and the counter electrode are tiled on different step surfaces respectively.

According to one aspect of the invention, the internal part is of a cylindrical or tapered structure, and the working electrode, the reference electrode and the counter electrode are insulated from each other around the surface of the internal part.

According to one aspect of the invention, at least two electrode groups are symmetrically arranged on two planes opposite the body portion.

According to one aspect of the invention, the thickness of the anti-interference layer is 0.1˜10 um.

According to one aspect of the invention, the thickness of the regulating layer is 1˜50 um.

According to one aspect of the invention, the biological compatibility layer is 1˜100 um thick.

According to one aspect of the invention, the predetermined condition is a preset time or electrode failure.

According to one aspect of the invention, the preset time is 1 to 14 days.

According to one aspect of the invention, the preset time is 14 days.

According to an aspect of the invention, if any electrode fails, the corresponding unfailed electrode with the same name takes over and enters the working state.

According to one aspect of the invention, each electrode set includes two working electrodes.

According to one aspect of the present invention, the substrate material is selected from one or more combinations of polytetrafluoroethylene, polyethylene, polyvinyl chloride, acrylonitrile-butadiene-styrene copolymer, polymethyl methacrylate, polycarbonate and polyimide.

Compared with the prior art, the technical scheme of the invention has the following advantages:

The invention discloses a micro analyte sensor, on the base of the internal part of the set has at least two electrode groups, each electrode group includes at least a working electrode and at least one additional electrodes, such sensor with two working electrode and at least two additional electrodes, electrode through a wire and set external part corresponding to the electrical connection PAD. The working electrode and the additional electrode are configured to pierce the host subskin, trigger according to predetermined conditions, and enter the working state alternately. At least two working electrodes and two additional electrodes can be used in place of the sensor, which prolongs the service life of the electrode relative to the sensor with a single electrode, and thus prolongs the service life of the sensor.

Further, the micro analyte sensor disclosed in the invention can be divided into a three-electrode system and a two-electrode system, wherein the three-electrode system consists of a counter electrode, a reference electrode and at least one working electrode, and the two-electrode system consists of a counter electrode and at least one working electrode. In addition, according to the number of working electrodes, the invention can also be divided into two situations: 1) single working electrode: there is only one working electrode. 2) Dual working electrode: there are two working electrodes, one of which is called “working electrode” for electroredox reaction with analyte to generate electrical signal, and the other is usually responsible for detecting the response signal of interference or background solution, called “auxiliary electrode”. All the electrode composition methods mentioned above have their own unique advantages, among which the three-electrode system can effectively control the detection potential, prevent potential drift, and improve the reliability of the parameter information of the detection analyte. However, the two-electrode system has simple structure and lower production cost.

Furthermore, the working electrode, the reference electrode and the counter electrode all contain at least electron conduction layer, anti-interference layer, enzyme layer, adjustment layer and biocompatible layer. The electron conduction layer is used to collect and conduct the electrons generated by the electroredox reaction between the electrode and the analyte. The anti-interference layer can prevent one or more interfering substances from penetrating into the electrolyte around the electrode and reacting with the electrode to generate interfering electrical signals. The enzyme layer is used for electroredox reaction with the analyte to be detected to produce electrons. The number of electrons produced varies according to the concentration of the analyte to be detected. The adjustment layer is mainly used to regulate the transmittance of oxygen and analyte transferred to the enzyme layer, so that the sensor can respond linearly to the change of analyte concentration. The biological compatibility layer is located at the outermost part of the electrode to eliminate the host's rejection of a foreign body and to reduce the formation of a shielding cell layer around the implanted electrode. The combination of the above functional layers makes each electrode eliminate the influence of possible interferences on the detection signal, adjust the diffusion performance of analyte and oxygen, protect the electrode, prolong the service life of the electrode, and improve the reliability of the parameter information of the detection analyte. Optimally, the thickness of the anti-interference layer is 0.1˜10 um, the thickness of the regulating layer is 1˜50 um, and the thickness of the biological compatibility layer is 1˜100 um.

Furthermore, each electrode is made of materials with good electrical conductivity and reinforcing inertia. The preferred electron conduction layers of the working electrode and the counter electrode are graphite, glassy carbon or noble metal, and the electron conduction layers of the reference electrode are Ag/AgCl or calomel. Considering the requirement of good ductility and stability of surface structure, precious metal materials such as gold, platinum and silver become the better choice. Preferably, the conductive layers of both the working electrode and the counter electrode are platinum.

Further, the enzyme layer is the glucose oxidase (GOX) layer, which allows the micro analyte sensor to detect glucose parameters in the host. The process of glucose oxidase action in the host is as follows:

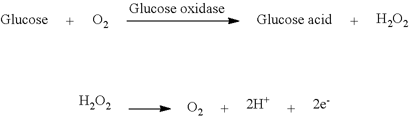

According to the different concentrations of glucose in the host body, the glucose oxidase layer can obtain different numbers of electrons accordingly, thus generating different current intensity. According to the current intensity information, the parameter information of glucose in the host body can be obtained.

Furthermore, the internal part is of planar structure, which is convenient for the installation of the electrode and the processing of the functional layer such as the anti-interference layer. Each electrode is insulated and tiled on the surface of the internal part to prevent the interference of the electrical signals between each other and improve the reliability of the parameter information of the analyte.

Further, the internal part to the ladder structure, flat out on the surface of the ladder that different for each electrode, on the one hand, the electrode spacing widening, reduces the interaction between electrode surface micro environment, ladder-like distribution of electrodes at the same time can effectively restrain interference of human reaction to the electrode response, on the other hand, distribution in different plane electrodes, under the premise that the effective area of each electrode remains the same, the width of the internal part can be further reduced. The smaller internal part size can reduce the rejection reaction of the host, prolong the service life of the electrode, and improve the reliability of the parameter information of the analyte.

Further, the internal part to the cylindrical structure, each electrode insulation to each other around the surface of the internal part, the internal part of the cylindrical structure reduces the sharp edge plane structure to the tissue of excitant, rejection is helpful to reduce the human body, prolong the service life of the electrode, improve the reliability of the detection of analytes parameter information.

Further, at least two electrodes set relative two symmetrically arranged in the internal part on the surface, on the one hand, symmetrical arrangement to facilitate the wires connected to the PAD go line, on the other hand, the body of the relative's two surfaces are used up, can in the limited area of the internal part of the arrangement more electrodes, more electrode to replace use, prolong the service life of the electrode.

Further, the electrode alternates into the working state under a predetermined trigger condition of a preset time or electrode failure. Preferably, the preset time is 1 to 14 days. For example, the preset time is 1 day. After every 1 day, another electrode group will be replaced and enter the working state, and different electrode groups will take over the work. Preferably, the preset time is 14 days. Generally, the effective working time of the single electrode is 14 days, and the service life of the single electrode is exhausted after 14 days. The preset time is 14 days, when the service life of the single electrode is exhausted, another set of electrodes can be automatically replaced to enter the working state. If any electrode fails in advance when the preset time is 14 days, the unfailed eponymous electrode in the other electrode group will replace it and enter the working state, so as to avoid the sensor failure due to the failure of one electrode and improve the reliability of the parameter information of the analyte detected.

Further, the substrate material of the sensor is selected from one or more combinations of polytetrafluoroethylene (TEFLON), polyethylene (PE), polyvinyl chloride (PVC), acrylonitrile-butadiene-styrene copolymer (ABS), polymethyl methacrylate (PMMA), polycarbonate (PC), polyimide (PI), etc. All of the above materials have excellent insulating properties, water impermeability and high mechanical strength, which can extend the service life of the sensor.

The second aspect of the present invention discloses a continuous analyte monitoring device, which comprises a bottom shell for mounting on the skin surface of the host. The sensor unit comprises a base and at least one micro analyte sensor as described above. The micro analyte sensor is fixed on the base, and the sensor unit is installed on the bottom shell through the base to detect the analyte parameter information in the host body. The transmitter unit comprises an internal circuit, an transmitter and an electrical connection area, and the electrical connection area is electrically connected with the sensor unit. The internal circuit stores predetermined conditions, and the transmitter is used to send the parameter information of the analyte to the outside world. A battery, which is used to provide electrical energy. And a receiver, which is used to receive analyte parameter information and indicate to the user.

The service life of the sensor is often the key factor to limit the service life of the continuous analyte monitoring device. The service life of the sensor is extended by the technical scheme of the replacement of multiple electrode groups, so the service life of the continuous analyte monitoring device is extended.

### BRIEF DESCRIPTION OF THE DRAWINGS

FIG. 1 is a top view of a planar structure of the sensor according to the embodiment of the invention.

FIG. 2 is a side view of the planar structure of the sensor as an embodiment of FIG. 1.

FIG. 3 is a sectional view of an electrode according to an embodiment of the invention.

FIG. 4 is a schematic diagram of function realization according to the embodiment of the invention.

FIG. 5 is a top view of the sensor with a stepped structure according to the embodiment of the invention.

FIG. 6 is a side view of the sensor with a stepped structure as an embodiment of FIG. 5.

FIG. 7 is a schematic diagram of the sensor having a cylindrical structure according to the embodiment of the invention.

FIG. 8 shows a V-V′ section view of the transducer with a cylindrical structure as an embodiment of FIG. 7.

FIG. 9 is a schematic diagram of a continuous analyte monitoring device according to an embodiment of the invention.

### DETAILED DESCRIPTION

As mentioned above, the service life of the existing technology of analyte sensor is not long, more than after the service life of the enzyme activity decreased, the parameter data reliability of the measured analyte will also decline, so in use after a certain period of time, users need to replace new sensors, new sensor into the host need to switch over a period of time, to formally enter the working state, cause the inconvenience on the use, but also increase the user's use cost.

In order to solve the problem, the present invention provides a micro analyte sensor, a plurality of electrode groups are arranged in the internal part of the sensor substrate, each group electrode includes at least one working electrode and at least one additional electrode, since all of the electrode group have been stabbed into the host, there is no hot-exchanging process, and each electrode body fluid environment is consistent, each electrode is configured to trigger in accordance with predetermined conditions when in use, and alternately enter the working state. The service life of each electrode can be superimposed to extend the service life of the sensor. At the same time, when any electrode fails in advance, the unfailed electrode with the same name can take its place and enter the working state, which improves the reliability of the parameter data of the analyte.

Various exemplary embodiments of the invention will now be described in detail with reference to the attached drawings. It is understood that, unless otherwise specified, the relative arrangement of parts and steps, numerical expressions and values described in these embodiments shall not be construed as limitations on the scope of the present invention.

In addition, it should be understood that the dimensions of the various components shown in the attached drawings are not necessarily drawn to actual proportions for ease of description, e.g. the thickness, width, length or distance of some elements may be enlarged relative to other structures.

The following descriptions of exemplary embodiments are illustrative only and do not in any sense limit the invention, its application or use. Techniques, methods and devices known to ordinary technicians in the relevant field may not be discussed in detail here, but to the extent applicable, they shall be considered as part of this Manual.

It should be noted that similar labels and letters indicate similar items in the appending drawings below, so that once an item is defined or described in one of the appending drawings, there is no need to discuss it further in the subsequent appending drawings.

In addition, it should be understood that one or more method steps referred to in the present invention do not exclude the possibility that other method steps may exist before and after the combined steps or that other method steps may be inserted between such explicitly mentioned steps, unless otherwise stated. It should also be understood that the combination connection between one or more devices/devices referred to in the invention does not preclude the existence of other devices/devices before and after the said combination devices/devices or the insertion of other devices/devices between the two specifically mentioned devices/devices, unless otherwise stated. And, unless otherwise specified, the serial number of the steps just a convenient tool for identifying the steps, rather than to limit the steps of the order or limit the scope of the present invention can be implemented, the relationship of the relative change or adjust, in the case of no substantial changes to technical content, when as well as the category of the present invention can be implemented.

### Implementation Example 1

### Planar Structure Sensor

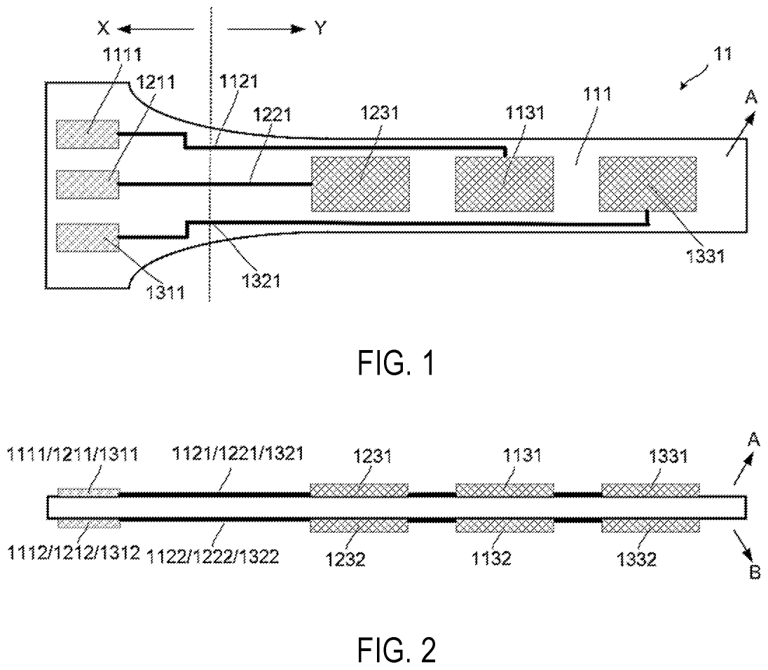

FIG. 1 is a top view of a planar structure of the sensor according to the embodiment of the invention. FIG. 2 is a side view of the planar structure of the sensor as an embodiment of FIG. 1.

Sensor 11 includes the substrate 111, which is divided into an external part X and an internal part Y as shown in FIG. 1 with dotted lines as the dividing line. Spread with the internal part Y electrode, including at least one working electrode 1131 and at least one additional electrode, obviously, in this example, additional electrode including a counter electrode 1231 and a reference electrode 1331, which constitute three electrodes system, the counter electrode 1231 is another pole relative to the working electrode 1131 and forms a closed loop with the working electrode 1131, so that the current on the electrode can be carried on normally. The reference electrode 1331 is used to provide the reference potential of the working electrode 1131, so that the detection potential can be effectively controlled. In this invention in another example, additional electrode can only include the counter electrode 1231, so as to form a two-electrode system, compared to the three-electrode system, the effective area of working electrode 1131 and counter electrode 1231 can be increased on the limited area of body part Y, so as to prolong the service life of the electrode, and because one electrode is removed, the process is simpler. However, working electrode 1131 does not have the detection potential of the reference electrode as a reference, so the reliability of the detection information of the analyte will be reduced. In another embodiment of the present invention, there are two working electrodes 1131, one of which produces an electrical signal by electroredox reaction with the analyte to be detected, and the other is used to detect the response signal of interference or background solution in the body fluid of the host, which is an auxiliary electrode.

Continuously refer to FIG. 1 and FIG. 2, the external part X is provided with PADs, which corresponds to the electrode one-to-one and is electrically connected through a wire, that is, the first PAD 1111 corresponding to the working electrode 1131 is electrically connected through wire 1121. The second PAD 1211 corresponding to the counter electrode 1231 is electrically connected through wire 1221. And the third PAD 1311 corresponding to the reference electrode 1331 is electrically connected through wire 1321. The different PADs, wires, and electrodes are insulated from each other to prevent interference with electrical signals.

Since sensor 11 is of planar structure, there are two opposite surfaces, namely surface A and surface B. The working electrode 1131, the counter electrode 1231 and the reference electrode 1331 are laid on the A surface of the sensor as an electrode group, in contrast, on the surface B, laid another electrode group, the electrode configuration can be a two-electrode system, can also be a three-electrode system, also can be double working electrode, optimization, consistent with electrode group on the surface A, which includes working electrode 1132, counter electrode 1232 and reference electrode 1332. Similarly, PAD is also laid on surface B, which corresponds to the electrode on surface B one-to-one, and is electrically connected through a wire. The fourth PAD 1112 corresponding to working electrode 1132 is electrically connected through wire 1122. The fifth PAD 1212 corresponding to the counter electrode 1232 is electrically connected through wire 1222. And the sixth PAD 1312 corresponding to the reference electrode 1332, electrically connected through wire 1322. In this way, if any electrode on surface A terminates its life or fails in advance, the same electrode on surface B can take over and enter the working state, improving the reliability of the parameter data of the detection analyte and prolonging the service life of the sensor.

Technicians in this field should understand that there is no restriction on the sequence or position of the PADs, conductors and electrodes laid on either surface A or B of the sensor. The PADs, wires, and electrodes on both surfaces may be symmetrically or asymmetrically arranged. The corresponding PAD, wire and electrode are laid on the same surface or can be laid on different surfaces. Preferably, the corresponding PAD, wire and electrode are laid on the same surface to facilitate the wiring of the wire. For example, the position of the working electrode 1131 on surface A can be changed with that of the counter electrode 1231 on surface A, or the position of the counter electrode 1231 on surface A can be changed with that of the reference electrode 1332 on surface B. No matter how the order and position of the PAD, wire and electrode on surface A and B change, as long as the PAD, wire and electrode exist one-to-one correspondence, each other can be insulated.

In other embodiments of the present invention, the service life of the sensor can be further extended by increasing the number of electrode groups by increasing the sensor area or decreasing the electrode area, although the planar structure sensor only has relative surface A and surface B. However, too large sensor area may increase the host's rejection reaction and cause the host's discomfort. Too small electrode area will reduce the sensitivity of the electrode and reduce the reliability of the detection parameters. An excessive number of electrode groups will also increase the complexity of the processing process, for example, the wiring of the wire will become very dense. Therefore, it is preferred that the number of electrode groups be two.

In other embodiments of the present invention, each electrode group may also be distributed on the same surface of the sensor, such as surface A or surface B, without limitation herein.

In the embodiment of the invention, the substrate 111 is a material with excellent insulating properties, mainly from inorganic non-metallic ceramics, silica glass and organic polymers, etc. At the same time, considering the application environment of implantable electrode, the substrate material is also required to have high water permeability and mechanical strength. Preferately, the substrate materials are selected from one or more combinations of polytetrafluoroethylene (Teflon), polyethylene (PE), polyvinyl chloride (PVC), acrylonitrile-butadiene-styrene copolymer (ABS), polymethyl methacrylate (PMMA), polycarbonate (PC), polyimide (PI), etc.

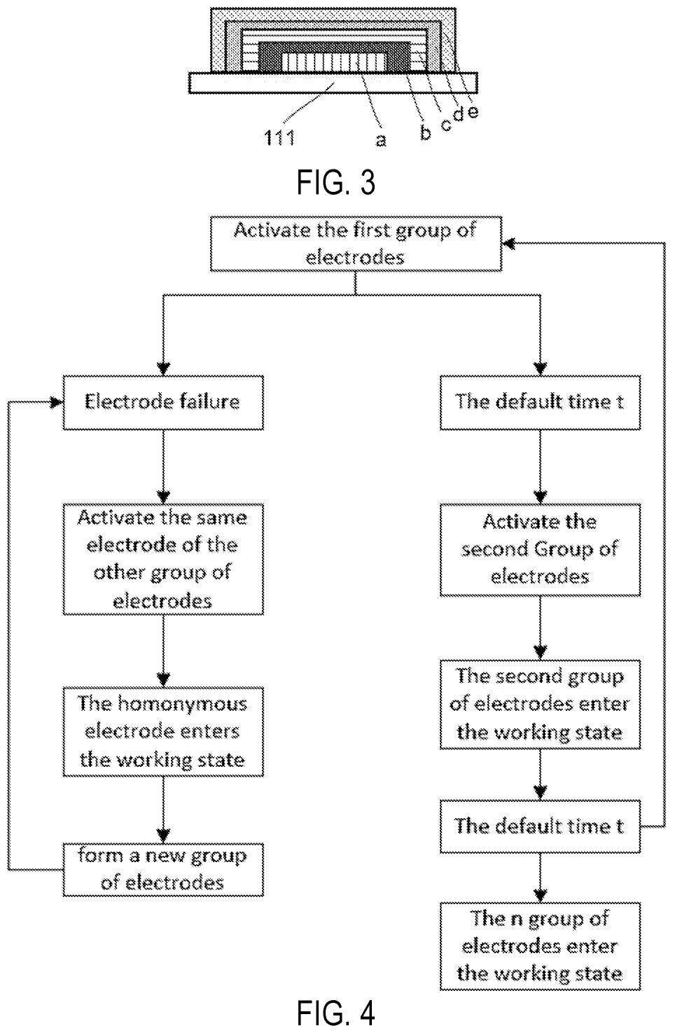

FIG. 3 shows a sectional view of the electrode. In one embodiment of the invention, the working electrode (auxiliary electrode), the counter electrode and the reference electrode comprise at least an electron conduction layer a, an anti-interference layer b, an enzyme layer c, an adjustment layer d and a biocompatible layer e.

### Electron Conduction Layer:

The electron conduction layer a is made of material with good electrical conductivity and fortification inertia. Preferred, the working electrode and the counter electrode are selected from graphite electrode, glass carbon electrode, noble metal and other materials, the reference electrode is selected from one of Ag/AgCl or calomel. Considering the requirements of good ductility and stability of surface structure, noble metal electrodes, such as gold electrode, platinum electrode and silver electrode, become a better choice. The working electrode and counter electrode are both platinum electrode for further optimization.

### Anti-Interference Layer:

The anti-interference layer b is located between the enzyme layer and the electron conduction layer. Interferers are molecules or substances that undergo electrochemical reduction or oxidation on the electrode surface, either directly or indirectly through an electron transfer agent, resulting in an erroneous signal that interferes with analyte detection. For example, for the determination of glucose as an analyte, common interferences in the body are urea, ascorbic acid, acetaminophen, and so on.

In the preferred example, the anti-interference layer b prevents one or more interference agents from penetrating the electrolyte surrounding the electrode. For example, the anti-interference layer b allows the analyte to be measured at the electrode (e.g., hydrogen peroxide) to pass through, while at the same time preventing the passage of other substances (e.g., potentially interfering substances). In a preferred scenario, the anti-interference layer b could be a very thin membrance designed to limit the diffusion of substances with molecular weights greater than 34 Da.

In another preferred example, the anti-interference layer b can be an organic polymer, which can be prepared from organosilane and a hydrophilic copolymer. Hydrophilic copolymers, preferably, polyethylene glycol (PEG), poly 2-hydroxyethyl methacrylate or polylysine. In a preferred embodiment, the thickness of the anti-interference layer b may range from 0.1 um or less to 10 um or more. The preferred thickness range is 0.5 um to 5 um.

### Enzyme Layer:

The enzyme layer c is coated with active enzymes. According to the type of analyte to be detected, the corresponding active enzymes are coated. Active enzymes can make the analyte to be detected produce some chemical reactions and generate electrons. According to different concentrations of analyte to be detected, the number of electrons produced is different, and the electrons are collected by the electron conduction layer, thus forming different current intensity. Therefore, current intensity information can be used to characterize the parameter information of the analyte.

Preferably, the enzyme layer c is coated with glucose oxidase (GOX).

### Adjustment Layers:

The adjustment layer d is located above the enzyme layer. In the embodiment of the present invention, when the enzyme layer is coated with glucose oxidase, the adjustment layer d is mainly used to regulate the transmittance of oxygen and glucose transferred to the enzyme layer. The amount of glucose (molar concentration) in body fluids is one order of magnitude higher than the amount of oxygen. However, for enzymatic sensors that require oxygen, an excess oxygen supply is needed to ensure that oxygen does not become a limiting substance, so that the sensor can respond linearly to changes in glucose concentration without being affected by oxygen partial pressure. In other words, when oxygen content is the limiting factor, the linear range of glucose oxygen monitoring reaction does not reach the expected concentration range. Without a semi-permeable membrane above the enzyme layer to regulate the passage of oxygen and glucose, the upper limit of the sensor's linear response to glucose is only about 40 mg/dL. However, in a clinical setting, the upper limit of the linear response of blood glucose levels needs to be about 500 mg/dL.

Adjustment layer d acts primarily as a semi-permeable membrane to regulate the amount of oxygen and glucose transmitted to the enzyme layer and, more specifically, to make oxygen excess a non-limiting factor. The upper limit of the linear response of the sensor to glucose with the adjustment layer can be reached to a higher level than that without the adjustment layer. In a preferred example, the ratio of oxygen-glucose transmittance in adjustment layer d can be reached to 200:1, thus ensuring that sufficient oxygen is available for the enzymatic reaction at any glucose and oxygen concentration that may be present subcutaneally.

In one preferred example, the adjustment layer d may be an organic polymer, which may be prepared from organosilane and a hydrophilic copolymer. Hydrophilic copolymer, preferably, copolymerization or graft of polyethylene glycol (PEG). Other hydrophilic copolymers that may be used include, but are not limited to, other diols such as propylene glycol, esters, amides, carbonates, and polypropylene glycol. The use of organosilicone polymers can obviously improve the oxygen transmission, and effectively control the glucose transmission. In a preferred implementation, the adjustment layer d may be in the thickness range of 1 um or less to 50 um or greater, with a preferred thickness range of 1 um to 10 um.

### Biological Compatibility Layer:

The biological compatibility layer e is located at the outermost part of the electrode, which is designed to eliminate the body's rejection of foreign bodies and reduce the formation of a shielding cell layer around the implanted electrode.

In a preferred example, the biological compatibility layer e can be prepared from organosilanes and a hydrophilic copolymer. Hydrophilic copolymer, preferably, copolymerization or graft of polyethylene glycol (PEG). Other hydrophilic copolymers that may be used include, but are not limited to, other diols such as propylene glycol, esters, amides, carbonates, and polypropylene glycol.

In a preferred embodiment, the thickness of the biological compatibility layer e may range from 1 um or less to 100 ums or more. A preferred thickness range is 10 um to 30 um.

In the embodiment of the invention, the thickness of base 11 is 0.01˜0.8 mm, each electrode is rectangular, the width of each electrode is 0.01˜1 mm, and the area is 0.1˜2 mm².

In other embodiments of the invention, the surface of each electrode is also provided with a modified layer of carbon nanotubes. Using carbon nanotubes unique mechanical strength, high specific surface area and chemical stability, fast electron transfer effect, in the shape of the surface of the electrode, via physical adsorption, embedding or covalent bond and way, such as to carbon nanotubes modified electrode surface in order to improve the electron transfer rate, at the same time, due to their large specific surface area can be as a kind of good catalyst carrier (enzyme). The modified carbon nanotube layer can be fixed on the electrode surface by nafion solution dispersion method, covalent fixation method, etc.

FIG. 4 is a schematic diagram of the functional realization of an embodiment of the invention.

After the sensor enters the host body, the internal circuit applies a voltage to the PAD, and the corresponding electrode of the PAD is activated to enter the working state. Generally speaking, after the electrode is activated, the effective working time is 1-14 days. After 14 days, the enzyme activity on the electrode decreases and it enters the failure state. At the same time, there may be electrode breakage or processing error and other reasons, and the activated electrode will enter the failure state in advance. If one group of electrodes is set on the sensor, once one of the electrodes enters the failure state, the sensor will fail, and the user needs to replace a new sensor, which reduces the user experience and increases the user's cost. If multiple groups of electrodes are set on the sensor, such as two groups of electrodes, once one electrode enters the failure state, the internal circuit will apply voltage to the PAD corresponding to the eponymous electrode of the other electrode group, activate the eponymous electrode and make it enter the working state to replace the failed electrode, so that the sensor can continue to work normally.

Specifically, refer to FIG. 1 and FIG. 2. After the sensor enters the host body, the first PAD 1111, the second PAD 1211 and the third PAD 1311 on surface A are firstly applied by the internal circuit, and the working electrode 1131, the counter electrode 1231 and the reference electrode 1331 on surface A enter the working state. Once one of the working electrode 1131, counter electrode 1231 and reference electrode 1331 fails in advance or its life terminates, the internal circuit switches to the PAD object that applies voltage, such as the working electrode 1131, fails in advance, the internal circuit switches to apply voltage to the fourth PAD 1112 on surface B to activate the working electrode 1132 on surface B. The new electrode group is combined with the unfailed counter electrode 1231 and reference electrode 1331 to detect the test analytes, so as to avoid the early failure of sensor 11, and the user does not need to replace the sensor due to the early failure of working electrode 1131, which enhances the user experience and reduces the user's cost of replacing the sensor.

It should be understood by technicians in the field that the above embodiments are not limited to the failure of the working electrode, and that the failure of other electrodes such as the counter electrode, the reference electrode, or the simultaneous failure of two or three electrodes may be replaced by the same electrode in the above embodiments.

Alternatively, the switch can be made before electrode failure or the end of life, in which case the predetermined condition is the preset time T. For example, the electrode fails after 14 days in normal working state, and the preset time T is 2 days. When the first electrode group is energized and works for 2 days, it switches to the second electrode group to energize, the second electrode group is activated, and the first electrode group is no longer energized and enters the sleep state. After working for 2 days in the second electrode group, the other electrode group can be activated, and the first electrode group can be activated again. This cycle of activation continues until the end of the service life of all electrode groups, all into the failure state. In this mode, the service life of multiple electrode groups is superimposed, thus extending the service life of the sensor.

Technicians in the field should understand that the preset time T can be any day up to 14 days, if due to the improvement of the electrode process or other reasons, its service life has been extended to n (n>14) days, the preset time T can be any day within n days.

### Implementation Example 2

### Stepped Structure Sensor

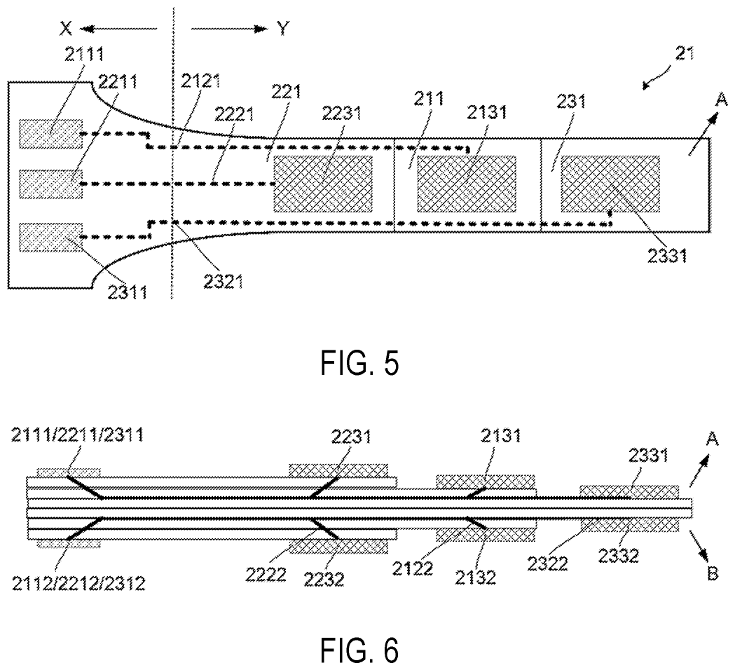

FIG. 5 is a top view of a stepped structure of the sensor in an embodiment of the invention. FIG. 6 is a side view of the sensor with a stepped structure as an embodiment of FIG. 5.

The stepped sensor 21 includes surface A and surface B, and each side is divided into the external part X and the internal part Y by the dotted line on the map. The internal part Y includes the first basement 211, the second basement 221 and the third basement 231, which form a stepped structure with each other. The number and level of the substrate are consistent with the number of electrodes on the surface. For example, when the surface A is a three-electrode system, the substrate is a three layer stepped structure. The substrate is a two-layer stepped structure when the surface A is a two-electrode system.

In the embodiment of the invention, the substrates of different levels are insulated from each other, and each electrode is electrically connected with the corresponding PAD through a wire distributed on a substrate (such as the third substrate), that is, part of the wire is in contact with the electrode, and the main part of the wire is located under the substrate, which can effectively protect the wire part. At the same time, each electrode is distributed on the substrate of different layers. On the one hand, the distance between the electrodes is widened to reduce the influence of the electrode surface microenvironment on each other. At the same time, the electrode distribution with step structure can effectively inhibit the interference of human reaction on the electrode response. On the other hand, by distributing the electrodes on different planes, the width of the whole sensor can be further reduced under the premise that the effective area of each electrode remains unchanged. The width of the stepped structure sensor can be reduced by about half on the basis of the planar structure sensor.

Correspondingly, surface B and surface A have a symmetric stepped structure. The external part X is provided with PADs, which corresponds to the electrode one-to-one, and is electrically connected through a wire, that is, the first PAD 2111 corresponding to the working electrode 2131 is electrically connected through wire 2121. The second PAD 2211 corresponding to the counter electrode 2231 is electrically connected through wire 2221. And the third PAD 2311 corresponding to the reference electrode 2331 is electrically connected through wire 2321. The different PADs, wires, and electrodes are insulated from each other to prevent interference with electrical signals.

The working electrode 2131, the counter electrode 2231 and the reference electrode 2331 are laid on the A surface of the sensor as an electrode group, in contrast, on the surface B, laid another electrode group, the electrode configuration can be a two-electrode system, can also be a three-electrode system, also can be double working electrode, optimization, consistent with surface A, which includes working electrode 2132, counter electrode 2232 and reference electrode 2332. Similarly, PADs are also laid on surface B, which corresponds to the electrode on surface B one-to-one, and is electrically connected through a wire, that is, the fourth PAD 2112 corresponding to working electrode 2132 is electrically connected through wire 2122. The fifth PAD 2212 corresponding to the counter electrode 2232 is electrically connected through wire 2222. And the sixth PAD 2312 corresponding to the reference electrode 2332 is electrically connected through wire 2322. In this way, if any electrode on surface A terminates its life or fails in advance, the same electrode on surface B can take over and enter the working state, improving the reliability of the parameter data of the detection analyte and prolonging the service life of the sensor.

Technicians in this field should understand that there is no restriction on the sequence or position of the PADs, conductors and electrodes laid on either surface A or B of the sensor. The PADs, wires, and electrodes on both surfaces may be symmetrically or asymmetrically arranged. The corresponding PAD, wire and electrode are laid on the same surface or can be laid on different surfaces. Preferably, the corresponding PAD, wire and electrode are laid on the same surface to facilitate the wiring of the wire. For example, the position of the working electrode 2131 on surface A can be changed with that of the counter electrode 2231 on surface A, or the position of the counter electrode 2231 on surface A can be changed with that of the reference electrode 2332 on surface B. No matter how the order and position of the PAD, wire and electrode on surface A and B change, as long as the PAD, wire and electrode exist one-to-one correspondence, each other can be insulated.

In other embodiments of the present invention, the service life of the sensor can be further extended by increasing the number of electrode groups by increasing the sensor area or decreasing the electrode area, although the stepped structure sensor only has relative surface A and B. However, too large sensor area may increase the host's rejection reaction and cause the host's discomfort. Too small electrode area will reduce the sensitivity of the electrode and reduce the reliability of the detection parameters. An excessive number of electrode groups will also increase the complexity of the processing process, for example, the wiring of the wire will become very dense. Therefore, it is preferred that the number of electrode group be two.

In other embodiments of the present invention, each electrode group may also be distributed on the same surface of the sensor, such as surface A or surface B, without limitation herein.

Case, the implementation of the present invention, the first basement 211, second basement 221 and the third basement 231 excellent insulation material, mainly from inorganic non-metallic ceramic, silica glass and organic polymer, etc., at the same time, considering the application environment, implantable electrodes also requires a basal material has high watertightness and mechanical strength. Preferately, the substrate materials are selected from one or more combinations of polytetrafluoroethylene (Teflon), polyethylene (PE), polyvinyl chloride (PVC), acrylonitrile-butadiene-styrene copolymer (ABS), polymethyl methacrylate (PMMA), polycarbonate (PC), polyimide (PI), etc.

In one embodiment of the invention, the working electrode (auxiliary electrode), the counter electrode and the reference electrode include at least an electron conduction layer a′, an anti-interference layer b′, an enzyme layer c′, an adjustment layer d′ and a biological compatibility layer e′.

### Electron Conduction Layer:

The electron conduction layer a′ is made of materials with good electrical conductivity and fortification inertia. Preferred, the working electrode and the counter electrode are selected from graphite electrode, glass carbon electrode, noble metal and other materials, the reference electrode is selected from one of Ag/AgCl or calomel. Considering the requirements of good ductility and stability of surface structure, noble metal electrodes, such as gold electrode, platinum electrode and silver electrode, become a better choice. The working electrode and counter electrode are both platinum electrode for further optimization.

### Anti-Interference Layer:

The anti-interference layer b′ is located between the enzyme layer and the electron conduction layer. Interferers are molecules or substances that undergo electrochemical reduction or oxidation on the electrode surface, either directly or indirectly through an electron transfer agent, resulting in an erroneous signal that interferes with analyte detection. For example, for the determination of glucose as an analyte, common interferences in the body are urea, ascorbic acid, acetaminophen, and so on.

In the preferred example, the anti-interference layer b′ prevents one or more interference agents from penetrating the electrolyte surrounding the electrode. For example, the anti-interference layer b′ allows the analyte to be measured at the electrode (e.g., hydrogen peroxide) to pass through, while at the same time preventing the passage of other substances (e.g., potentially interfering substances). In a preferred scenario, the anti-interference layer b′ could be a very thin membrance designed to limit the diffusion of substances with molecular weights greater than 34 Da.

In another preferred example, the anti-interference layer b′ can be an organic polymer, which can be prepared from organosilane and a hydrophilic copolymer. Hydrophilic copolymers, preferably, polyethylene glycol (PEG), poly (2-hydroxyethyl methacrylate) and poly (lysine). In a preferred embodiment, the thickness of the anti-interference layer b′ may range from 0.1 um or less to 10 um or more. The preferred thickness range is 0.5 um to 5 um.

### Enzyme Layer:

The enzyme layer c′ is coated with active enzymes. According to the type of analyte to be detected, the corresponding active enzymes are coated. Active enzymes can make the analyte to be detected produce some chemical reactions and generate electrons. According to different concentrations of analyte to be detected, the number of electrons produced is different, and the electrons are collected by the electron conduction layer, thus forming different current intensity. Therefore, current intensity information can be used to characterize the parameter information of the analyte.

Preferably, the enzyme layer c is coated with glucose oxidase (GOX).

### Adjustment Layers:

The adjustment layer d′ is located above the enzyme layer. In the embodiment of the present invention, when the enzyme layer is coated with glucose oxidase, the adjustment layer d is mainly used to regulate the transmittance of oxygen and glucose transferred to the enzyme layer.

The amount of glucose (molar concentration) in body fluids is one order of magnitude higher than the amount of oxygen. However, for enzymatic sensors that require oxygen, an excess oxygen supply is needed to ensure that oxygen does not become a limiting substance, so that the sensor can respond linearly to changes in glucose concentration without being affected by oxygen partial pressure. In other words, when oxygen content is the limiting factor, the linear range of glucose oxygen monitoring reaction does not reach the expected concentration range. Without a semi-permeable membrane above the enzyme layer to regulate the passage of oxygen and glucose, the upper limit of the sensor's linear response to glucose is only about 40 mg/dL. However, in a clinical setting, the upper limit of the linear response of blood glucose levels needs to be about 500 mg/dL.

Adjustment layer d′ acts primarily as a semi-permeable membrane to regulate the amount of oxygen and glucose transmitted to the enzyme layer and, more specifically, to make oxygen excess a non-limiting factor. The upper limit of the linear response of the sensor to glucose with the adjustment layer can be reached to a higher level than that without the adjustment layer. In a preferred example, the ratio of oxygen-glucose transmittance in adjustment layer d′ can be reached to 200:1, thus ensuring that sufficient oxygen is available for the enzymatic reaction at any glucose and oxygen concentration that may be present subcutaneally.

In one preferred example, the adjustment layer d′ may be an organic polymer, which may be prepared from organosilane and a hydrophilic copolymer. Hydrophilic copolymer, preferably, copolymerization or graft of polyethylene glycol (PEG). Other hydrophilic copolymers that may be used include, but are not limited to, other diols such as propylene glycol, esters, amides, carbonates, and polypropylene glycol. The use of organosilicone polymers can obviously improve the oxygen transmission, and effectively control the glucose transmission. In a preferred implementation, the adjustment layer d′ may be in the thickness range of 1 um or less to 50 um or greater, with a preferred thickness range of 1 um to 10 um.

### Biological Compatibility Layer:

The biological compatibility layer e′ is located at the outermost part of the electrode, which is designed to eliminate the body's rejection of foreign bodies and reduce the formation of a shielding cell layer around the implanted electrode.

In a preferred example, the biological compatibility layer e′ can be prepared from organosilanes and a hydrophilic copolymer. Hydrophilic copolymer, preferably, copolymerization or graft of polyethylene glycol (PEG). Other hydrophilic copolymers that may be used include, but are not limited to, other diols such as propylene glycol, esters, amides, carbonates, and polypropylene glycol.

In a preferred embodiment, the thickness of the biological compatibility layer e′ may range from 1 um or less to 100 ums or more. A preferred thickness range is 10 um to 30 um.

In the embodiment of the invention, the thickness of the first basement 211, the second basement 221 and the third basement 231 is 0.01-0.8 mm, the width of each electrode is 0.01-1 mm, and the area is 0.1-2 mm².

In other embodiments of the invention, the surface of each electrode is also provided with a modified layer of carbon nanotubes. Using carbon nanotubes unique mechanical strength, high specific surface area and chemical stability, fast electron transfer effect, in the shape of the surface of the electrode, via physical adsorption, embedding or covalent bond and way, such as to carbon nanotubes modified electrode surface in order to improve the electron transfer rate, at the same time, due to their large specific surface area can be as a kind of good catalyst carrier (enzyme). The modified carbon nanotube layer can be fixed on the electrode surface by Nafion solution dispersion method, covalent fixation method, etc.

In the embodiments of the invention, the realization method of the function of electrode replacement of different electrode groups to prolong the service life of the sensor is consistent with that of embodiments 1, and will not be repeated here.

### Example 3

### Cylindrical Structure Sensor

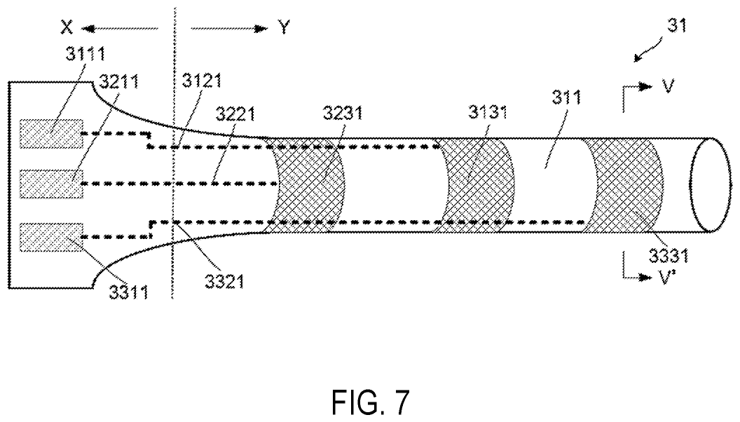

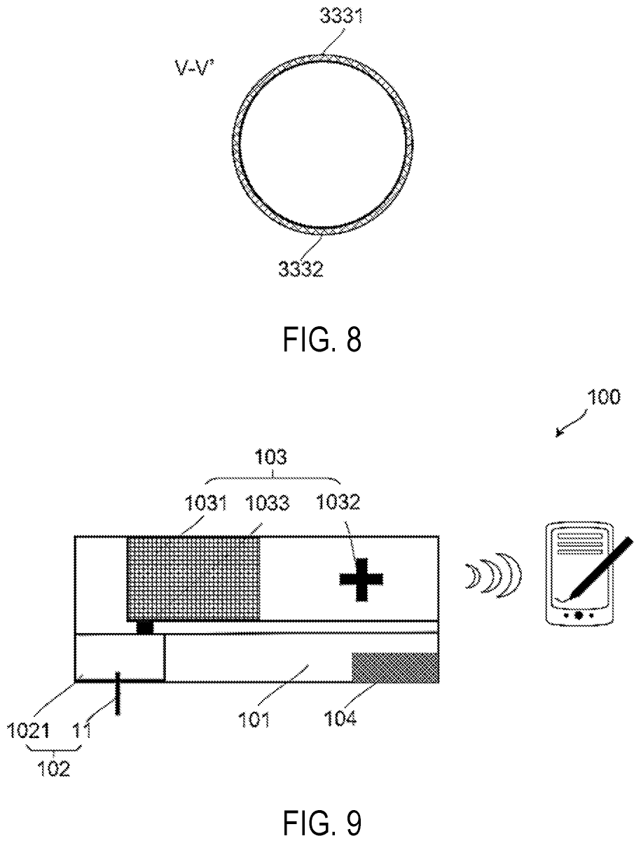

FIG. 7 is a schematic diagram of the cylindrical structure of the sensor in the embodiment of the invention. FIG. 8 shows a V-V′ section view of the transducer with a cylindrical structure as an embodiment of FIG. 7.

Sensor 31 with a cylindrical structure is demarcated by a dotted line on the figure, and its substrate 311 is divided into an external part X and an internal part Y. The external X part is planar or cylindrical, preferably planar. The internal part Y includes substrate 311, which is cylindrical, and each electrode is surrounded on the surface of the base. Compared with the flat electrode, the ring electrode has no sharp edge, which reduces the irritation to human tissue and the rejection reaction of human body, which is conducive to the realization of implantable long-term detection and improves the service life of the sensor.

The internal part Y includes at least one working electrode 3131 and at least one additional electrode. Obviously, in this embodiment, the additional electrode includes counter electrode 3231 and reference electrode 3331, thus forming a three-electrode system. The counter electrode 3231 is another pole relative to the working electrode 3131 and forms a closed loop with the working electrode 3131. The reference electrode 3331 is used to provide the reference potential of the working electrode 3131, so the detection potential can be effectively controlled. In this invention in another example, additional electrode can only include a counter electrode 3231, so as to form a two-electrode system, compared to the three-electrode system, the effective area of working electrode 3131 and counter electrode 3231 can be increased on the limited area of internal part Y, thus the service life of the electrode is extended, and the processing process is simpler because there is no electrode. But the working electrode 3131 does not have the detection potential of the reference electrode as a reference, the reliability of the detection information of the analyte will be reduced. In another embodiment of the present invention, there are two working electrodes 3131, one of which produces an electrical signal by electroredox reaction with the analyte to be detected, and the other is used to detect the response signal of interference or background solution in the body fluid of the host, which is an electric auxiliary electrode.

Continue to refer to FIG. 7, the external part X is provided with a PAD, which corresponds to the electrode one-to-one, and is electrically connected through a wire, that is, the first PAD 3111 corresponding to the working electrode 3131 is electrically connected through wire 3121. The second PAD 3211 corresponding to the counter electrode 3231 is electrically connected through wire 3221. And the third PAD 3311 corresponding to the reference electrode 3331, which is electrically connected through wire 3321, the working electrode 3131, the counter electrode 3231 and the reference electrode 3331 constitute an electrode group. The different PADs, wires, and electrodes are insulated from each other to prevent interference with electrical signals.

Each electrode is laid on the internal part Y in a semi-enclosed way, so that two electrodes can be placed in the same place to form an enclosure on the internal part Y. Specifically, according to FIG. 8, reference electrodes 3331 and 3332 are semicircle rings at V-V′ of internal part Y, whose inner diameter is equal to the outer diameter of internal part Y, and they are insulated from each other to maximize the use of the surface area of internal part Y.

In other embodiments of the invention, the enclosure formed with reference electrode 3331 May be the working electrode 3131 or the counter electrode 3231 of the same electrode group, or the working electrode (not shown in the figure) or the counter electrode (not shown in the figure) of other electrode groups, so that in the case of termination of service life or premature failure of any electrode, the corresponding electrode of the same name can be replaced into the working state to improve the reliability of the parameter data of the analyte and prolong the service life of the sensor.

Technicians in this field should understand that there is no restriction on the sequence or location of PADs, conductors, and electrodes laid on substrate 311. PAD, wire and electrode can be arranged symmetrically or asymmetrically. No matter how the sequence and position of PAD, wire and electrode change, the one-to-one correspondence and insulation relationship between PAD, wire and electrode can be made.

In other embodiments of the present invention, the number of electrode groups can also be increased by increasing the sensor area or decreasing the electrode area, thus further increasing the service life of the sensor. However, too large sensor area may increase the host's rejection reaction and cause the host's discomfort. Too small electrode area will reduce the sensitivity of the electrode and reduce the reliability of the detection parameters. An excessive number of electrode groups will also increase the complexity of the processing process, for example, the wiring of the wire will become very dense. Therefore, it is preferred that the number of electrode groups be two.

In the embodiment of the invention, the substrate 311 is a material with excellent insulating properties, mainly from inorganic non-metallic ceramics, silica glass and organic polymers, etc. At the same time, considering the application environment of implantable electrode, the substrate material is also required to have high water permeability and mechanical strength. Preferately, the substrate materials are selected from one or more combinations of polytetrafluoroethylene (Teflon), polyethylene (PE), polyvinyl chloride (PVC), acrylonitrile-butadiene-styrene copolymer (ABS), polymethyl methacrylate (PMMA), polycarbonate (PC), polyimide (PI), etc.

In the embodiment of the present invention, the outer diameter of the inner part Y of substrate 311 and the inner diameter of the electrode are 0.01˜100 um, preferably 10˜50 um. Electrodes may be half ring, ⅓ ring, ¼ ring, or other scale rings.

In one embodiment of the invention, the working electrode (auxiliary electrode), the counter electrode and the reference electrode include at least an electron conduction layer a″, an anti-interference layer b″, an enzyme layer c″, an adjustment layer d″ and a biocompatible layer e″.

### Electron Conduction Layer:

The electron conduction layer a″ is made of materials with good electrical conductivity and fortification inertia. Preferred, the working electrode and the counter electrode are selected from graphite electrode, glass carbon electrode, noble metal and other materials, the reference electrode is selected from one of Ag/AgCl or calomel. Considering the requirements of good ductility and stability of surface structure, noble metal electrodes, such as gold electrode, platinum electrode and silver electrode, become a better choice. The working electrode and counter electrode are both platinum electrode for further optimization.

### Anti-Interference Layer:

The anti-interference layer b″ is located between the enzyme layer and the electron conduction layer. Interferers are molecules or substances that undergo electrochemical reduction or oxidation on the electrode surface, either directly or indirectly through an electron transfer agent, resulting in an erroneous signal that interferes with analyte detection. For example, for the determination of glucose as an analyte, common interferences in the body are urea, ascorbic acid, acetaminophen, and so on.

In the preferred example, the anti-interference layer b″ prevents one or more interference agents from penetrating the electrolyte surrounding the electrode. For example, the anti-interference layer b″ allows the analyte to be measured at the electrode (e.g., hydrogen peroxide) to pass through, while at the same time preventing the passage of other substances (e.g., potentially interfering substances). In a preferred scenario, the anti-interference layer b″ could be a very thin membrance designed to limit the diffusion of substances with molecular weights greater than 34 Da.

In another preferred example, the anti-interference layer b can be an organic polymer, which can be prepared from organosilane and a hydrophilic copolymer. Hydrophilic copolymers, preferably, polyethylene glycol (PEG), poly (2-hydroxyethyl methacrylate) and poly (lysine). In a preferred embodiment, the thickness of the anti-interference layer b″ may range from 0.1 um or less to 10 um or more. The preferred thickness range is 0.5 um to 5 um.

### Enzyme Layer:

The enzyme layer c″ is coated with active enzymes. According to the type of analyte to be detected, the corresponding active enzymes are coated. Active enzymes can make the analyte to be detected produce some chemical reactions and generate electrons. According to different concentrations of analyte to be detected, the number of electrons produced is different, and the electrons are collected by the electron conduction layer, thus forming different current intensity. Therefore, current intensity information can be used to characterize the parameter information of the analyte.

Preferably, the enzyme layer c″ is coated with glucose oxidase (GOX).

### Adjustment Layers:

The adjustment layer d″ is located above the enzyme layer. In the embodiment of the present invention, when the enzyme layer is coated with glucose oxidase, the adjustment layer d″ is mainly used to regulate the transmittance of oxygen and glucose transferred to the enzyme layer. The amount of glucose (molar concentration) in body fluids is one order of magnitude higher than the amount of oxygen. However, for enzymatic sensors that require oxygen, an excess oxygen supply is needed to ensure that oxygen does not become a limiting substance, so that the sensor can respond linearly to changes in glucose concentration without being affected by oxygen partial pressure. In other words, when oxygen content is the limiting factor, the linear range of glucose oxygen monitoring reaction does not reach the expected concentration range. Without a semi-permeable membrane above the enzyme layer to regulate the passage of oxygen and glucose, the upper limit of the sensor's linear response to glucose is only about 40 mg/dL. However, in a clinical setting, the upper limit of the linear response of blood glucose levels needs to be about 500 mg/dL.

Adjustment layer d″ acts primarily as a semi-permeable membrane to regulate the amount of oxygen and glucose transmitted to the enzyme layer and, more specifically, to make oxygen excess a non-limiting factor. The upper limit of the linear response of the sensor to glucose with the adjustment layer can be reached to a higher level than that without the adjustment layer. In a preferred example, the ratio of oxygen-glucose transmittance in adjustment layer d″ can be reached to 200:1, thus ensuring that sufficient oxygen is available for the enzymatic reaction at any glucose and oxygen concentration that may be present subcutaneally.

In one preferred example, the adjustment layer d″ may be an organic polymer, which may be prepared from organosilane and a hydrophilic copolymer. Hydrophilic copolymer, preferably, copolymerization or graft of polyethylene glycol (PEG). Other hydrophilic copolymers that may be used include, but are not limited to, other diols such as propylene glycol, esters, amides, carbonates, and polypropylene glycol. The use of organosilicone polymers can obviously improve the oxygen transmission, and effectively control the glucose transmission. In a preferred implementation, the adjustment layer d″ may be in the thickness range of 1 um or less to 50 um or greater, with a preferred thickness range of 1 um to 10 um.

### Biological Compatibility Layer:

The biological compatibility layer e″ is located at the outermost part of the electrode, which is designed to eliminate the body's rejection of foreign bodies and reduce the formation of a shielding cell layer around the implanted electrode.

In a preferred example, the biological compatibility layer e″ can be prepared from organosilanes and a hydrophilic copolymer. Hydrophilic copolymer, preferably, copolymerization or graft of polyethylene glycol (PEG). Other hydrophilic copolymers that may be used include, but are not limited to, other diols such as propylene glycol, esters, amides, carbonates, and polypropylene glycol.

In a preferred embodiment, the thickness of the biological compatibility layer e″ may range from 1 um or less to 100 ums or more. A preferred thickness range is 10 um to 30 um.

In other embodiments of the invention, the surface of each electrode is also provided with a modified layer of carbon nanotubes. Using carbon nanotubes unique mechanical strength, high specific surface area and chemical stability, fast electron transfer effect, in the shape of the surface of the electrode, via physical adsorption, embedding or covalent bond and way, such as to carbon nanotubes modified electrode surface in order to improve the electron transfer rate, at the same time, due to their large specific surface area can be as a kind of good catalyst carrier (enzyme). The modified carbon nanotube layer can be fixed on the electrode surface by Nafion solution dispersion method, covalent fixation method, etc.

In the embodiments of the invention, the realization method of the function of electrode replacement of different electrode groups to prolong the service life of the sensor is consistent with that of embodiments 1, and will not be repeated here.

The field technicians, understandably, sensor internal part Y is not necessarily limited to the shape of the above three cases, for example, in other cases, may be circular, round ring, conical, such as spiral shape, and the arrangement of electrodes on the shape is based on the shape of a internal part, which only electrodes can facilitate laid on the internal part, there is no restriction here.

FIG. 9 is a schematic diagram of the continuous analyte monitoring device 100 in an embodiment of the invention. The continuous analyte monitoring device 100 includes a chassis 101 for mounting on the skin surface of the host. The sensor unit 102 comprises a base 1021 and a micro analyte sensor 11 (21/31) as described above. The micro analyte sensor 11 (21/31) is fixed on the base, and the sensor unit 102 is installed on the bottom shell 101 through the base. The transmitter unit 103 comprises an internal circuit 1031, an transmitter 1032 and an electrical connection area 1033. The electrical connection area 1033 is electrically connected with the sensor unit 102. The internal circuit 1031 stores the predetermined conditions of the electrode switching described above, and the transmitter 1032 is used to send the analytical parameter information to the outside world. Battery 104, battery 104 is used to provide electricity. Receiver 105, receiver 105 is used to receive analyte parameter information and indicate to the user.

To sum up, the invention discloses a kind of micro analyte sensor, how much is set in the internal part of the sensor base electrode groups, each group of electrode group includes at least a working electrode and at least one additional electrode, when each electrode is configured to use, according to the predetermined conditions to trigger, alternately into the working state, the service life of the electrode can be stacked, prolong the service life of the sensor. At the same time, when any electrode fails in advance, the unfailed electrode with the same name can take its place and enter the working state, which improves the reliability of the measured parameter data of the analyte and enhances the user experience.

The present invention also made public a kind of use as stated earlier the micro continuous analyte sensor analytes monitoring devices, due to the continuous service life of the analyte detection device is often limited to the service life of the sensor, micro analyte sensor adopted as stated earlier, can prolong the service life of continuous analyte detection equipment, to enhance the user experience, reduce the user's use cost.

Although some specific embodiments of the invention have been detailed through examples, technicians in the field should understand that the above examples are for illustrative purposes only and are not intended to limit the scope of the invention. Persons skilled in the field should understand that the above embodiments may be modified without departing from the scope and spirit of the present invention. The scope of the invention is limited by the attached claims.

1. A micro analyte sensor, comprising:
a substrate comprising an internal part and an external part;
at least two electrode groups, located on a surface of the internal part, wherein each of the electrode groups comprises at least one working electrode and at least one additional electrode;
a plurality of pads, wherein each of the pads corresponding to one of the electrode groups is arranged in the external part, and each of the pads is electrically connected with the working electrode and the additional electrode of the one of the electrode groups through a wire,
the working electrode and the additional electrode of each one of the electrode groups are configured to trigger and enter a working state alternately when in use.

2. According to the micro analyte sensor mentioned in claim 1, wherein the additional electrode includes a counter electrode.

3. According to the micro analyte sensor mentioned in claim 2, wherein the additional electrode includes a reference electrode.

4. According to the micro analyte sensor mentioned in claim 3, wherein each of the working electrode, the reference electrode and the counter electrode at least include an electron conduction layer, an anti-interference layer, an enzyme layer, an adjustment layer and abiological compatible layer.

5. According to the micro analyte sensor mentioned in claim 4, wherein each of the electron conduction layer of the working electrode and the electron conduction layer of the counter electrode is one of graphite, glassy carbon or noble metal.

6. According to the micro analyte sensor mentioned in claim 5, wherein each of the electron conduction layer of the working electrode and the electron conduction layer of the counter electrode is platinum.

7. According to the micro analyte sensor mentioned in claim 4, wherein the electron conduction layer of the reference electrode is one of Ag/AgCl or calomel.

8. (canceled)

9. According to the micro analyte sensor mentioned in claim 3, wherein the internal part is a plane structure, and the working electrode, the reference electrode and the counter electrode are insulated and tiled on the surface of the internal part.

10. According to the micro analyte sensor mentioned in claim 3, wherein the internal part is a stepped structure, and the working electrode, the reference electrode and the counter electrode are tiled on different step surfaces respectively.

11. According to the micro analyte sensor mentioned in claim 3, wherein the internal part is a cylindrical structure, and the working electrode, the reference electrode and the counter electrode are insulated from each other around the surface of the internal part.

12. According to the micro analyte sensor mentioned in claim 1, wherein the at least two electrode groups are symmetrically arranged on two planes of a body part opposite to each other.

13. According to the micro analyte sensor mentioned in claim 4, wherein a thickness of the anti-interference layer is 0.1˜10 um.

14. According to the micro analyte sensor mentioned in claim 4, wherein a thickness of the adjustment layer is 1˜50 um.

15. According to the micro analyte sensor mentioned in claim 4, wherein a thickness of the biological compatible layer is 1˜100 um.

16. According to the micro analyte sensor mentioned in claim 1, wherein when one of the electrode groups meets a condition, another one of the electrode groups is triggered.

17. According to the micro analyte sensor mentioned in claim 16, wherein the condition is when the one of the electrode groups is in the working state in 1˜14 days.

18. According to the micro analyte sensor mentioned in claim 17, wherein the condition is when the one of the electrode groups is in the working state for 14 days.

19. According to the micro analyte sensor mentioned in claim 16, wherein the condition is when the one of the electrode groups fails.

20. According to the micro analyte sensor mentioned in claim 1, wherein the at least one working electrode includes two working electrodes.

21. (canceled)

22. A continuous analyte monitoring device, comprising:
a bottom shell stalled on a surface of a host;
a sensor unit comprising a base and at least one micro analyte sensor according to claim 1, wherein the micro analyte sensor is fixed on the base and the sensor unit is installed on the bottom shell through the base to detect analyte parameter information in the host;
a transmitter unit comprising an internal circuit, a transmitter and an electrical connection area, wherein the electrical connection area is electrically connected with the sensor unit, and the transmitter is configured to output the analyte parameter information;
a battery configured to provide electric energy; and
a receiver configured to receive the analyte parameter information and to display.
